SEQUENCE LISTING (58 sequences)

SEQ ID NO: 1 - DNA, 56 bp, Escherichia coli
acaacacattttactgctgtgaactttgttgtaatcctgcctgtgctggatgttat

SEQ ID NO: 2 - protein, 19 aa, Escherichia coli
NNTFYCCELCCNPACAGCY

SEQ ID NO: 3 - protein, 18 aa, Escherichia coli
NTFYCCELCCYPACAGCN

SEQ ID NO: 4 - DNA, 56 bp, Escherichia coli
atagtagcaattactgctgtgaattgtgttgtaatcctgcttgtaacgggtgctat

SEQ ID NO: 5 - protein, 19 aa, Escherichia coli
NSSNYCCELCCNPACNGCY

SEQ ID NO: 6 - protein, 15 aa, Rattus norvegicus
PNTCEICAYAACTGC

SEQ ID NO: 7 - protein, 18 aa, Artificial Sequence
  peptide fragment 1
NNTFYCCELCCNPACAGC

SEQ ID NO: 8 - protein, 17 aa, Artificial sequence
  peptide  fragment 2
NTFYCCELCCNPACAGC

SEQ ID NO: 9 - protein, 16 aa, Artificial Sequence
  peptide  fragment 3
TFYCCELCCNPACAGC

SEQ ID NO: 10 - protein, 15 aa, Artificial sequence
  peptide fragment 4
FYCCELCCNPACAGC

SEQ ID NO: 11 - protein, 14 aa, Artificial sequence
  peptide  fragment 5
YCCELCCNPACAGC

SEQ ID NO: 12 - protein, 13 aa, Artificial sequence
  peptide fragment 6
CCELCCNPACAGC

SEQ ID NO: 13 - protein, 18 aa, Artificial sequence
  peptide fragment 7
NTFYCCELCCNPACAGCY

SEQ ID NO: 14 - protein, 17 aa, Artificial sequence
  peptide fragment 8
TFYCCELCCNPACAGCY

SEQ ID NO: 15 - protein, 16 aa, Artificial sequence
  peptide fragment 9
FYCCELCCNPACAGCY

SEQ ID NO: 16 - protein, 15 aa, Artificial sequence
  peptide fragment 10
YCCELCCNPACAGCY

SEQ ID NO: 17 - protein, 14 aa, Artificial sequence
  peptide fragment 11
CCELCCNPACAGCY

SEQ ID NO: 18 - protein, 17 aa, Artificial sequence
  peptide fragment 12
NTFYCCELCCYPACAGC

SEQ ID NO: 19 - protein, 16 aa, Artificial sequence
  peptide fragment 13
TFYCCELCCYPACAGC

SEQ ID NO: 20 - protein, 15 aa, Artificial sequence
  peptide fragment 14
FYCCELCCYPACAGC

SEQ ID NO: 21 - protein, 14 aa, Artificial sequence
  peptide fragment 15
YCCELCCYPACAGC

SEQ ID NO: 22 - protein, 13 aa, Artificial sequence
  peptide fragment 16
CCELCCYPACAGC

SEQ ID NO: 23 - protein, 17 aa, Artificial sequence
  peptide fragment 17
TFYCCELCCYPACAGCN

SEQ ID NO: 24 - protein, 16 aa, Artificial sequence
  peptide fragment 18
FYCCELCCYPACAGCN

SEQ ID NO: 25 - protein, 15 aa, Artificial sequence
  peptide fragment 19
YCCELCCYPACAGCN

SEQ ID NO: 26 - protein, 14 aa, Artificial sequence
  peptide fragment 20
CCELCCYPACAGCN

SEQ ID NO: 27 - protein, 18 aa, Artificial sequence
  peptide fragment 21
NSSNYCCELCCNPACTGC

SEQ ID NO: 28 - protein, 17 aa, Artificial sequence
  peptide fragment 22
SSNYCCELCCNPACTGC

SEQ ID NO: 29 - protein, 16 aa, Artificial sequence
  peptide fragment 23
SNYCCELCCNPACTGC

SEQ ID NO: 30 - protein, 15 aa, Artificial sequence
  peptide fragment 24
NYCCELCCNPACTGC

SEQ ID NO: 31 - protein, 14 aa, Artificial sequence
  peptide fragment 25
YCCELCCNPACTGC

SEQ ID NO: 32 - protein, 13 aa, Artificial sequence
  peptide fragment 26
CCELCCNPACTGC

SEQ ID NO: 33 - protein, 18 aa, Artificial sequence
  peptide fragment 27
SSNYCCELCCNPACTGCY

SEQ ID NO: 34 - protein, 17 aa, Artificial sequence
  peptide fragment 28
SNYCCELCCNPACTGCY

SEQ ID NO: 35 - protein, 16 aa, Artificial sequence
  peptide fragment 29
NYCCELCCNPACTGCY

SEQ ID NO: 36 - protein, 15 aa, Artificial sequence
  peptide fragment 30
YCCELCCNPACTGCY

SEQ ID NO: 37 - protein, 14 aa, Artificial sequence
  peptide fragment 31
CCELCCNPACTGCY

SEQ ID NO: 38 - protein, 18 aa, Artificial sequence
  peptide fragment 32
NTFYCCELCCNPACAGCY

SEQ ID NO: 39 - protein, 18 aa, Artificial sequence
  peptide fragment 33
NTFYCCELCCAPACAGCY

SEQ ID NO: 40 - protein, 18 aa, Artificial sequence
  peptide fragment 34
NTFYCCELCCNAACAGCY

SEQ ID NO: 41 - protein, 17 aa, Artificial sequence
  peptide fragment 35
NTFYCCELCCNPACAGC

SEQ ID NO: 42 - protein, 15 aa, Artificial sequence
  peptide fragment 36
YCCELCCNPACAGCY

SEQ ID NO: 43 - protein, 14 aa, Artificial sequence
  peptide fragment 37
YCCELCCNPACAGC

SEQ ID NO: 44 - protein, 14 aa, Artificial sequence
  peptide fragment 38
CCELCCNPACAGCY

SEQ ID NO: 45 - protein, 13 aa, Artificial sequence
  peptide fragment 39
CCELCCNPACAGC

SEQ ID NO: 46 - protein, 25 aa, Yersinia enterocolitica
QACDPPSPPAEVCCDVCCNPACAGC

SEQ ID NO: 47 - protein, 16 aa, Vibrio cholerae
IDCCICCNPACFGCLN

SEQ ID NO: 48 - protein, 18 aa, Yersinia enterocolitica
SSDWDCCDVCCNPACAGC

SEQ ID NO: 49 - protein, 19 aa, Artificial sequence
  peptide fragment 40
NSSNYCCELCCYPACTGCY

SEQ ID NO: 50 - protein, 13 aa, Artificial sequence
  peptide fragment 41
CCDVCCNPACTGC

SEQ ID NO: 51 - protein, 14 aa, Artificial sequence
  peptide fragment 42
CCDVCCYPACTGCY

SEQ ID NO: 52 - protein, 14 aa, Artificial sequence
  peptide fragment 43
CCDLCCNPACAGCY

SEQ ID NO: 53 - protein, 14 aa, Artificial sequence
  peptide fragment 44
CCQLCCNPACTGCY

SEQ ID NO: 54 - protein, 15 aa, Homo sapiens
PGTCEICAYAACTGC

SEQ ID NO: 55 - protein, 15 aa, Didelphis virginiana
QEDCELCINVACTGC

SEQ ID NO: 56 - protein, 16 aa, Artificial sequence
  peptide fragment 45
NDDCELCVNVACTGCL

SEQ ID NO: 57 - protein, 94 aa, Homo sapiens
VTVQDGNFSFSLESVKKLKDLQEPQEPRVGKLRNFAPIPGEPVVPILCSNPNFPEELKPLCKEPNAQEILQRLEEIAEDPGTCEICAYAACTGC

SEQ ID NO: 58 - protein, 86 aa, Homo sapiens
VYIQYQGFRVQLESMKKLSDLEAQWAPSPRLQAQSLLPAVCHHPALPQDLQPVCASQEASSIFKTLRTIANDDCELCVNVACTGCL

(19) United States
(12) Patent Application Publication
(10) Pub. No.: US 2012/0064039 A1
(43) Pub. Date: Mar. 15, 2012

(54) Use of Guanylyl Cyclase C Agonists to Suppress Appetite

(75) Inventors: Michael Valentino, Philadelphia, PA (US); Jeiru Egeria Lin, Philadelphia, PA (US); Scott A. Waldman, Ardmore, PA (US)

(73) Assignee: THOMAS JEFFERSON UNIVERSITY, Philadelphia, PA (US)

(21) Appl. No.: 13/203,041

(22) PCT Filed: Feb. 24, 2010
(86) PCT No.: PCT/US10/25277

§ 371 Date: Nov. 27, 2011

Related U.S. Application Data
(60) Provisional application No. 61/155,052, filed on Feb. 24, 2009.

(51) Int. Cl. A61K 38/17; A61P 3/00; A61P 1/12; A61K 35/74

(52) U.S. Cl. 424/93.4; 514/4.9

(57) ABSTRACT

Compositions comprising a guanylyl cyclase C agonist formulated for sustained specific release of an amount effective to suppress appetite when administered orally to an individual are disclosed. Compositions comprising a guanylyl cyclase C agonist formulated for intravenous delivery in an amount effective to suppress appetite when administered intravenously to an individual are also disclosed. Methods of suppressing appetite in an individual are disclosed.

### BACKGROUND OF THE INVENTION

While it is known that biochemical signals are involved in hunger and satiety, much is unknown concerning the hormones and other messengers involved. The regulation of messages to and from the gastrointestinal track, the brain and other organs of the body is not completely understood. One messenger involved is cholecystokinin (CCK) whereby agonists to receptors of CCK may be involved in satiety. Large amounts of resources are devoted to research to identify active agents which affect CCK or other hormones or messengers involved, in hunger and satiety. Such agents may be useful to suppress hunger and/or confer a feeling of satiety in individuals such that such individuals may reduce their calorie intake or overall food intake.

Reduction in calorie intake is important in both losing body weight as well as in preventing the gaining of body weight, particularly in those individuals who have previously lost body weight and are at risk for weight regain. Overweight, obesity and morbid obesity refer to conditions involving excessive body weight, particularly excessive body fat, which leads to or exacerbates numerous health problems. Creation of caloric deficit, i.e. calorie use exceeds calorie intake, is the basis for weight loss, and appetite suppression can be a useful tool in the compliance to any restriction on calorie intake. Weight regain is a common occurrence among individuals who have successfully lost weight. Upon achieving a weight loss goal, individuals return to prior eating and exercise patterns and weight returns. Appetite suppression can be a useful tool in the prevention of weight regain by assisting an individual in the adoption of eating patterns which limit calorie intake. Athletes and fitness enthusiasts who have low body fat percentage are often required to reduce body fat percentage further. Such individuals must carefully control and balance the nutrition they ingest when attempting to eliminate body fat without having a negative effect on muscle quantity and performance. Appetite suppression can be a useful tool in controlling nutritional uptake.

Guanylyl cyclase C (GCC) is a cell receptor reported to be present in the brush border cells of the bowel in humans. The coding sequence of human gene is disclosed in Genbank Accession Number NM_{—}004963, which is incorporated herein by reference.

Guanylin and uroguanylin are native ligands of GCC. The ligands are small peptides which bind to GCC and have agonist activity.

The heat stable enterotoxin produced by E. coli, referred to as ST, binds to GCC as well. ST binding to GCC is at a much higher affinity and the result is diarrhea. E. coli that produces ST is responsible for what is known as travelers diarrhea. Among infants, elderly and other vulnerable individuals, the diarrhea caused by ST can be lethal.

The expression of GCC by colorectal cancer cells make it a useful market to detect and target metastatic colorectal cancer. In addition, GCC agonists have been disclosed for use in the treatment of primary colorectal cancer, and autoimmune diseases such as inflammatory bowel disease. Such GCC agonists includes anti-GCC antibodies, ST peptides and related toxins, guanylin, uroguanylin and modified forms of such peptides. The delivery of GCC agonists to the bowel is useful in the prevention of formation of polyps, in the prevention of development of polyps in colorectal cancer, in the treatment of primary colorectal cancer, in the treatment of autoimmune diseases involving the colon such as inflammatory bowel disease.

Although it has been speculated that GCC agonists increase gastrointestinal motility and are therefore potentially useful to treat obesity by moving food through the bowel more quickly and thereby decreasing caloric absorption, GCC agonists have not been linked to appetite and satiety signaling.

There remains a need for more effective compositions to suppress appetite and reduce calorie and food intake.

### SUMMARY OF THE INVENTION

The present invention also relates to compositions comprising a guanylyl cyclase C agonist in an amount effective to suppress appetite when administered orally to an individual.

The present invention relates to methods of suppressing appetite comprising orally administered to an individual in need thereof an effective amount of guanylyl cyclase C agonist.

The present invention relates to methods of reducing body weight comprising orally administered to an individual in need thereof an amount of guanylyl cyclase C agonist effective to suppress appetite and reduce calorie or food uptake.

The present invention relates to methods of treating an overweight individual comprising orally administered to an individual in need thereof an amount of guanylyl cyclase C agonist effective to suppress appetite and reduce calorie or food uptake.

The present invention relates to methods of preventing weight regain in an individual who is at risk of regaining previously reduced body weight comprising orally administered to an individual in need thereof an amount of guanylyl cyclase C agonist effective to suppress appetite and reduce calorie or food uptake.

### DESCRIPTION OF PREFERRED EMBODIMENTS

As used herein the terms guanylyl cyclase C agonist and “GCC agonists” are used interchangeably and refer to molecules which bind to guanylyl cyclase C and thereby induce its activity.

As used herein “overweight” refers to body weights considered overweight, obese or morbidly obese.

Administration of a GCC agonist refers to administration of one or more compounds that bind to and activate GCC.

Suppression of appetite refers to reducing an individual's feelings of hunger and desire to consume food. Suppression of appetite may result from activation of neurochemical pathway which when activated results in an individual experiencing a feeling of satiety such their hunger and desire to consume food is reduced.

Guanylyl cyclase C (GCC) is a cellular receptor expressed by cells lining the large and small intestines. The binding of GCC agonists to GCC in the gastrointestinal track is known to activate GCC, leading to an increase in intracellular cGMP, which results in activation of downstream signaling events.

The binding of GCC agonists to GCC in the gastrointestinal track may effect the neurochemical pathway of hunger and satiety. The satiety signal is may be reduced or eliminated in the absence of GCC activation in individuals. GCC agonists can be administered to individuals in amounts that effect hunger and satiety signals such that the individual's appetite is suppressed.

While not intending to be limited to any theory, there may be a correlation between guanylin expression and high fat intact which provides mechanisms for suppression of appetite to prevent obesity and thereby prevent the increased risks of cancer and other diseases associated with obesity. High fat diets lead to a decrease in guanylin present in the intestines as well as a decrease in proguanylin and/or prouroguanylin in the blood. A deficiency of guanylin levels in the intestine that occurs in obese individuals results in a decrease in satiety. Thus, the consumption of high fat foods becomes part of a self-reinforcing loop by which the reduced guanylin levels interfere with an individual's satiety/appetite suppression mechanisms, which leads to more eating, and the reduction of guanylin that occurs. The individuals who experience this in the cycle frequently often become obese. Obesity increases the risk of colorectal cancer and other diseases. The suppression of guanylin may be linked to both the loss of guanylyl cyclase C signaling as an event in the transformation process and to the cycle that includes the mechanism which results in down modulation of guanylin expression and obesity.

GCC agonists are known. Two native GCC agonists, guanylin and uroguanylin, have been identified (see U.S. Pat. Nos. 5,969,097 and 5,489,670, which are each incorporated herein by reference. In addition, several small peptides, which are produced by enteric pathogens, are toxigenic agents which cause diarrhea (see U.S. Pat. No. 5,518,888, which is incorporated herein by reference). The most common pathogen derived GCC agonist is the heat stable entertoxin produced by strains of pathogenic E. coli. Native heat stable enterotoxin produced by pathogenic E coli is also referred to as ST. A variety of other pathogenic organisms including Yersinia and Enterobacter, also make enterotoxins which can bind to guanylyl cyclase C in an agonistic manner. In nature, the toxins are generally encoded on a plasmid which can “jump” between different species. Several different toxins have been reported to occur in different species. These toxins all possess significant sequence homology, they all bind to ST receptors and they all activate guanylate cyclase, producing diarrhea.

ST has been both cloned and synthesized by chemical techniques. The cloned or synthetic molecules exhibit binding characteristics which are similar to native ST. Native ST isolated from E. coli is 18 or 19 amino acids in length. The smallest “fragment” of ST which retains activity is the 13 amino acid core peptide extending toward the carboxy terminal from cysteine 6 to cysteine 18 (of the 19 amino acid form). Analogues of ST have been generated by cloning and by chemical techniques. Small peptide fragments of the native ST structure which include the structural determinant that confers binding activity may be constructed. Once a structure is identified which binds to ST receptors, non-peptide analogues mimicking that structure in space are designed.

U.S. Pat. Nos. 5,140,102 and 7,041,786, and U.S. Published Applications US 2004/0258687 A1 and US 2005/0287067 A1 also refer to compounds which may bind to and activate guanylyl cyclase C.

SEQ ID NO:1 discloses a nucleotide sequence which encodes 19 amino acid ST, designated ST Ia, reported by So and McCarthy (1980) Proc. Natl. Acad. Sci. USA 77:4011, which is incorporated herein by reference.

The amino acid sequence of ST Ia is disclosed in SEQ ID NO:2.

SEQ ID NO:3 discloses the amino acid sequence of an 18 amino acid peptide which exhibits ST activity, designated ST I*, reported by Chan and Giannella (1981) J. Biol. Chem. 256:7744, which is incorporated herein by reference.

SEQ ID NO:4 discloses a nucleotide sequence which encodes 19 amino acid ST, designated ST Ib, reported by Mosely et al. (1983) Infect. Immun. 39:1167, which is incorporated herein by reference.

The amino acid sequence of ST Ib is disclosed in SEQ ID NO:5.

A 15 amino acid peptide called guanylin which has about 50% sequence homology to ST has been identified in mammalian intestine (Currie, M. G. et al. (1992) Proc. Natl. Acad. Sci. USA 89:947-951, which is incorporated herein by reference). Guanylin binds to ST receptors and activates guanylate cyclase at a level of about 10- to 100-fold less than native ST. Guanylin may not exist as a 15 amino acid peptide in the intestine but rather as part of a larger protein in that organ. The amino acid sequence of guanylin from rodent is disclosed as SEQ ID NO:6.

SEQ ID NO:7 is an 18 amino acid fragment of SEQ ID NO:2. SEQ ID NO:8 is a 17 amino acid fragment of SEQ ID NO:2. SEQ ID NO:9 is a 16 amino acid fragment of SEQ ID NO:2. SEQ ID NO:10 is a 15 amino acid fragment of SEQ ID NO:2. SEQ ID NO:11 is a 14 amino acid fragment of SEQ ID NO:2. SEQ ID NO:12 is a 13 amino acid fragment of SEQ ID NO:2. SEQ ID NO:13 is an 18 amino acid fragment of SEQ ID NO:2. SEQ ID NO:14 is a 17 amino acid fragment of SEQ ID NO:2. SEQ ID NO:15 is a 16 amino acid fragment of SEQ ID NO:2. SEQ ID NO:16 is a 15 amino acid fragment of SEQ ID NO:2. SEQ ID NO:17 is a 14 amino acid fragment of SEQ ID NO:2.

SEQ ID NO:18 is a 17 amino acid fragment of SEQ ID NO:3. SEQ ID NO:19 is a 16 amino acid fragment of SEQ ID NO:3. SEQ ID NO:20 is a 15 amino acid fragment of SEQ ID NO:3. SEQ ID NO:21 is a 14 amino acid fragment of SEQ ID NO:3. SEQ ID NO:22 is a 13 amino acid fragment of SEQ ID NO:3. SEQ ID NO:23 is a 17 amino acid fragment of SEQ ID NO:3. SEQ ID NO:24 is a 16 amino acid fragment of SEQ ID NO:3. SEQ ID NO:25 is a 15 amino acid fragment of SEQ ID NO:3. SEQ ID NO:26 is a 14 amino acid fragment of SEQ ID NO:3.

SEQ ID NO:27 is an 18 amino acid fragment of SEQ ID NO:5. SEQ ID NO:28 is a 17 amino acid fragment of SEQ ID NO:5. SEQ ID NO:29 is a 16 amino acid fragment of SEQ ID NO:5. SEQ ID NO:30 is a 15 amino acid fragment of SEQ ID NO:5. SEQ ID NO:31 is a 14 amino acid fragment of SEQ ID NO:5. SEQ ID NO:32 is a 13 amino acid fragment of SEQ ID NO:5. SEQ ID NO:33 is an 18 amino acid fragment of SEQ ID NO:5. SEQ ID NO:34 is a 17 amino acid fragment of SEQ ID NO:5. SEQ ID NO:35 is a 16 amino acid fragment of SEQ ID NO:5. SEQ ID NO:36 is a 15 amino acid fragment of SEQ ID NO:5. SEQ ID NO:37 is a 14 amino acid fragment of SEQ ID NO:5.

SEQ ID NO:27, SEQ ID NO:31, SEQ ID NO:36 AND SEQ ID NO:37 are disclosed in Yoshimura, S., et al. (1985) FEBS Lett. 181:138, which is incorporated herein by reference.

SEQ ID NO:38, SEQ ID NO:39 and SEQ ID NO:40, which are derivatives of SEQ ID NO:3, are disclosed in Waldman, S. A. and O'Hanley, P. (1989) Infect. Immun. 57:2420, which is incorporated herein by reference.

SEQ ID NO:41, SEQ ID NO:42, SEQ ID NO:43, SEQ ID NO:44 and SEQ ID NO:45, which are a derivatives of SEQ ID NO:3, are disclosed in Yoshimura, S., et al. (1985) FEBS Lett. 181:138, which is incorporated herein by reference.

SEQ ID NO:46 is a 25 amino acid peptide derived from Y. enterocolitica which binds to the ST receptor.

SEQ ID NO:47 is a 16 amino acid peptide derived from V. cholerae which binds to the ST receptor. SEQ ID NO:47 is reported in Shimonishi, Y., et al. FEBS Lett. 215:165, which is incorporated herein by reference.

SEQ ID NO:48 is an 18 amino acid peptide derived from Y. enterocolitica which binds to the ST receptor. SEQ ID NO:48 is reported in Okamoto, K., et al. Infec. Immun. 55:2121, which is incorporated herein by reference.

SEQ ID NO:49, is a derivative of SEQ ID NO:5.

SEQ ID NO:50, SEQ ID NO:51, SEQ ID NO:52 and SEQ ID NO:53 are derivatives. SEQ ID NO:54 is the amino acid sequence of guanylin from human.

A 15 amino acid peptide called uroguanylin has been identified in mammalian intestine from opossum (Hamra, S. K. et al. (1993) Proc. Natl. Acad. Sci. USA 90:10464-10468, which is incorporated herein by reference; see also Forte L. and M. Curry 1995 FASEB 9:643-650; which is incorporated herein by reference). SEQ ID NO:55 is the amino acid sequence of uroguanylin from opossum.

A 16 amino acid peptide called uroguanylin has been identified in mammalian intestine from human (Kita, T. et al. (1994) Amer. J. Physiol. 266:F342-348, which is incorporated herein by reference; see also Forte L. and M. Curry 1995 FASEEB 9:643-650; which is incorporated herein by reference). SEQ ID NO:56 is the amino acid sequence of uroguanylin from human.

SEQ ID NO:57 is the amino acid sequence of proguanylin, a guanylin precursor which is processed into active guanylin.

SEQ ID NO:58 is the amino acid sequence of prouroguanylin, a uroguanylin precursor which is processed into active uroguanylin.

Although proguanylin and prouroguanylin are precursors for mature guanylin and mature uroguanylin respectively, they may be used as GCC agonists as described herein provide they are delivered such that they can be processed into the mature peptides.

U.S. Pat. Nos. 5,140,102, 7,041,786 and 7,304,036, and U.S. Published Applications US 2004/0258687, US 2005/0287067, 20070010450, 20040266989, 20060281682, 20060258593, 20060094658, 20080025966, 20030073628, 20040121961 and 20040152868, which are each incorporated herein by reference, also refer to compounds which may bind to and activate guanylyl cyclase C.

In addition to human guanylin and human uroguanylin, guanylin or uroguanylin may be isolated or otherwise derived from other species such as cow, pig, goat, sheep, horse, rabbit, bison, etc. Such guanylin or uroguanylin may be administered to individuals including humans.

Antibodies including GCC binding antibody fragments can also be GGC agonists. Antibodies may include for example polyclonal and monoclonal antibodies including chimeric, primatized or humanized monoclonal antibodies as well as antibody fragments that bind to GGC with agonist activity such as CDRs, FAbs, F(Ab), Fv's including single chain Fv and the like. Antibodies may be IgE, IgA or IgM for example.

### Effective Amount

In order to use GCC agonists to suppress appetite, an effective amount must be delivered. As used herein, an “effective amount” is an amount which will suppress an individual's appetite following administration for at least a period of 2 hours. Suppression of appetite is characterized by reduced sensations of hunger and/or a reduction in the severity of such sensations and/or a reduction in the desire to eat and/or a reduction in the severity of a desire to eat and/or a feeling of fullness or satiety and/or an increase in the feeling of fullness or satiety. As a result, individuals with suppressed appetites generally reduce food intake or caloric intake.

In some embodiments, an initial loading dose and/or multiple administrations are required for an initial effect to be observed. Thereafter, regular administrations are required to maintain the suppressed condition for a period of time. The GCC agonists administered to the individual may have an additive effect with the satiety signals present naturally in the individual. Likewise, the effect induced by GCC agonists administered to the individual may be counteracted by the hunger signals present naturally in the individual. The affinity that the GCC agonists have to GCC may also effect the effect such GCC agonists have on satiety and hunger.

In some embodiments, an individual is administered a course of GCC agonists sufficient to sustain the appetite suppression effect over an extended period of time, such as a for example 3 days, 7 days, 14 days, 21 days, 28 days or more. To achieve such extended effect, multiple doses may be administered at levels and in intervals such that the amount of GCC agonist present, either free or bound to GCC, remains above the threshold needed to be effective to suppress appetite.

Generally, GCC agonists are administered in an amount ranging from 100 ug to 1 gram every 4-48 hours. In some embodiments, GCC agonists are administered in an amount ranging from 1 mg to 750 mg every 4-48 hours. In some embodiments, GCC agonists are administered in an amount ranging from 10 mg to 500 mg every 4-48 hours. In some embodiments, GCC agonists are administered in an amount ranging from 50 mg to 250 mg every 4-48 hours. In some embodiments, GCC agonists are administered in an amount ranging from 75 mg to 150 mg every 4-48 hours, In some embodiments, the GCC agonists are administered prior to eating, e.g prior to mealtime, at a time such that the GCC agonist will have its effects on satiety and appetite before or during eating in order to reduce caloric consumption. In some embodiments, the GCC agonists are administered 30 minutes to 4 hours prior to eating, 30 minutes to 3 hours prior to eating, 30 minutes to 2 hours prior to eating, 30 minutes to 1 hour prior to eating, 1-4 hours prior to eating, 1-3 hours prior to eating, 1-2 hours prior to eating, 2-4 hours prior to eating, or 3-4 hours prior to eating. In some embodiments, the GCC agonists are administered prior to eating, e.g prior to mealtime, at a time such that the GCC agonist will have its effects on satiety and appetite before or during eating in order to reduce caloric consumption. In some embodiments, individuals are administered a GCC agonist in a range of 2.5 mg-250 mg intravenously. In some embodiments, individuals are administered a GCC agonist in a range of 5 mg-200 mg intravenously. In some embodiments, individuals are administered a GCC agonist in a range of 10 mg-150 mg intravenously. In some embodiments, individuals are administered a GCC agonist in a range of 15 mg-100 mg intravenously. In some embodiments, individuals are administered a GCC agonist in a range of 5 mg-250 mg intravenously. In some embodiments, individuals are administered a GCC agonist in a range of 10 mg-250 mg intravenously. In some embodiments, individuals are administered a GCC agonist in a range of 15 mg-250 mg intravenously. In some embodiments, individuals are administered a GCC agonist in a range of 20 mg-250 mg intravenously.

In some embodiments, doses are administered every 4 or more hours. In some embodiments, doses are administered every 6 or more hours. In some embodiments, doses are administered every 8 or more hours. In some embodiments, doses are administered every 12 or more hours. In some embodiments, doses are administered every 24 or more hours. In some embodiments, doses are administered every 48 or more hours. In some embodiments, doses are administered every 4 hours or less. In some embodiments, doses are administered every 6 hours or less. In some embodiments, doses are administered every 8 hours or less. In some embodiments, doses are administered every 12 hours or less. In some embodiments, doses are administered every 24 hours or less. In some embodiments, doses are administered every 48 hours or less.

In some embodiments, the formulation and/or dosage administered and/or frequency of administration is selected to minimize the negative side effects associated with GCC activation, i.e. cramping and diarrhea. In some embodiments, the amount of GCC agonists available to activate GCC is controlled such that the individual experiences minimal effects leading to diarrhea or cramping/intestinal contractions-increased motity, or none at all. In some embodiments, additives or co-agents are administered in combination with GCC agonists to a minimize diarrhea or cramping/intestinal contractions-increased motity. For example, the individual may be administered a compound that before, simultaneously or after administration with a compound that relieves diarrhea. Such anti-diarrheal component may be incorporated in the formulation. Anti-diarrheal compounds and preparations, such as loperamide, bismuth subsalicylate and probiotic treatments such as strains of Lactobaccilus, are well known and widely available.

### Route

Current methods for delivering GCC agonists to the colorectal track involved the oral delivery of such GCC agonists. ST peptides and other GCC agonist peptides, for example, are stable and can survive the stomach acid and pass through the small intestine to the colorectal track. While they can reach the colorectal track intact and able to positively interact with cells that express GCC, their presence in the small intestine can have serious side effects. Moreover, regular dosing is required to maintain an effective level of GCC agonist in the colorectal track, such dosing further exacerbating the side effects caused by the GCC agonists passing through the small intestine.

The ST receptor ligand is administered to the individual by any route that will allow for the delivery of the ligand to cells that express ST receptors. For example, in some embodiments, the ST receptor ligand is administered orally. In some embodiments, the ST receptor ligand is administered parenterally. In some embodiments, the ST receptor ligand is administered into the circulatory system of the individual. In some embodiments, the ST receptor ligand is administered intravenously. In some embodiments, the ST receptor ligand is administered subcutaneously.

GCC agonists such as for example ST, guanylin and uroguanylin, can survive the gastric environment. Thus, they may be administered without coating or protection against stomach acid. However, in order to more precisely control the release of GCC agonists administered orally, the GCC agonist may be enterically coated so that some or all of the GCC agonist is released after passing through the stomach. Such enteric coating may also be designed to provide a sustained or extended release of the GCC agonist over the period of time with which the coated GCC agonist passes through the intestines.

Most enteric coatings are intended to protect contents from stomach acid. Accordingly, they are designed to release active agent upon passing through the stomach. The coatings and encapsulations used herein are provided to begin releasing the GCC agonist in the small intestine and preferably over an extended period of time so that GCC agonist concentrations can be maintained t an effective level for a greater period of time.

According to some embodiments, the GCC agonists are coated or encapsulated with a sufficient amount of coating material that the time required for the coating material to dissolve and release the GCC agonists corresponds with the time required for the coated or encapsulated composition to travel from the mouth to intestines.

According to some embodiments, the GCC agonists are coated or encapsulated with coating material that does not fully dissolve and release the GCC agonists until it comes in contact with conditions present in the small intestine. Such conditions may include the presence of enzymes in the colorectal track, pH, tonicity, or other conditions that vary relative to the stomach.

According to some embodiments, the GCC agonists are coated or encapsulated with coating material that is designed to dissolve in stages as it passes from stomach to small intestine to large intestine.

According to some embodiments, the GCC agonists are complexed with another molecular entity such that they are inactive until the GCC agonists cease to be complexed with molecular entity and are present in active form. In such embodiments, the GCC agonists are administered as “prodrugs” which become processed into active GCC agonists in the colorectal track.

Examples of technologies which may be used to formulate GCC agonists for sustained release when administered orally include, but are not limited to: U.S. Pat. Nos. 5,007,790, 4,451,260, 4,132,753, 5,407,686, 5,213,811, 4,777,033, 5,512,293, 5,047,248 and 5,885,616.

Examples of technologies which may be used to formulate GCC agonists or inducers for large intestine specific release when administered include, but are not limited to: U.S. Pat. No. 5,108,758 issued to Allwood, et al. on Apr. 28, 1992 which discloses delayed release formulations; U.S. Pat. No. 5,217,720 issued to Sekigawa, et al. on Jun. 8, 1993 which discloses coated solid medicament form having releasability in large intestine; U.S. Pat. No. 5,541,171 issued to Rhodes, et al. on Jul. 30, 1996 which discloses orally administrable pharmaceutical compositions; U.S. Pat. No. 5,688,776 issued to Bauer, et al. on Nov. 18, 1997 which discloses crosslinked polysaccharides, process for their preparation and their use; U.S. Pat. No. 5,846,525 issued to Maniar, et al. on Dec. 8, 1998 which discloses protected biopolymers for oral administration and methods of using same; U.S. Pat. No. 5,863,910 to Bolonick, et al. on Jan. 26, 1999 which discloses treatment of chronic inflammatory disorders of the gastrointestinal tract; U.S. Pat. No. 6,849,271 to Vaghefi, et al. on Feb. 1, 2005 which discloses microcapsule matrix microspheres, absorption-enhancing pharmaceutical compositions and methods; U.S. Pat. No. 6,972,132 to Kudo, et al. on Dec. 6, 2005 which discloses a system for release in lower digestive tract; U.S. Pat. No. 7,138,143 to Mukai, et al. Nov. 21, 2006 which discloses coated preparation soluble in the lower digestive tract; U.S. Pat. No. 6,309,666; U.S. Pat. No. 6,569,463, U.S. Pat. No. 6,214,378; U.S. Pat. No. 6,248,363; U.S. Pat. No. 6,458,383, U.S. Pat. No. 6,531,152, U.S. Pat. No. 5,576,020, U.S. Pat. No. 5,654,004, U.S. Pat. No. 5,294,448, U.S. Pat. No. 6,309,663, U.S. Pat. No. 5,525,634, U.S. Pat. No. 6,248,362, U.S. Pat. No. 5,843,479, and U.S. Pat. No. 5,614,220, which are each incorporated herein by reference.

In some embodiments, individuals are administered a GCC agonist intravenously. In some embodiments individuals are administered a peptide GCC agonist intravenously. In some embodiments individuals are administered SEQ ID NOsS:1-56, prouroguanylin (SEQ ID NO:57) or uroguanylin (SEQ ID NO:58), intravenously.

In some embodiments, individuals are administered a GCC agonist using an implanted depot or an pump similar to an insulin pump. The GCC agonist is released and taken up by the blood. In some embodiments individuals are administered a peptide GCC agonist intravenously. In some embodiments individuals are administered SEQ ID NOsS:1-56, prouroguanylin (SEQ ID NO:57) or uroguanylin (SEQ ID NO:58), intravenously.

### Expression in Gut Bacteria

According to some aspects of the invention, innocuous bacteria of species that normally populate the colon are provided with genetic information needed to produce a guanylyl cyclase C agonist in the colon, making such guanylyl cyclase C agonist available to produce the effect of activating the guanylyl cyclase C on colon cells, or inhibiting formation of colon polyps, or treating colon polyps, or inhibiting formation of colorectal tumors, or treating colorectal cancer. The existence of a population of bacteria which can produce guanylyl cyclase C agonist provides a continuous administration of the guanylyl cyclase C agonist in the site where it is needed. In some embodiments, the nucleic acid sequences that encode the guanylyl cyclase C agonist are under the control of an inducible promoter. Accordingly, the individual may turn expression on or off depending upon whether or not the inducer is ingested. In some embodiments, the inducer is formulated to be specifically released in the colon, thereby preventing induction of expression by the bacteria that may be populating other sites such as the small intestine. In some embodiments, the bacteria are is sensitive to a particular drug or auxotrophic such that it can be eliminated by administration of the drug or withholding an essential supplement.

The technology for introducing expressible forms of genes into bacteria is well known and the materials needed are widely available.

In some embodiments, bacteria which comprise coding sequences for a GCC agonist may be those of a species which commonly inhabits the intestinal track of an individual. Common gut flora include species from the genera Bacteroides, Clostridium, Fusobacterium, Eubacterium, Ruminococcus, Peptococcus, Peptostreptococcus, Bifidobacteriu, Escherichia and Lactobacillus. In some embodiments, the bacteria is selected from a strain known to be useful as a probiotic. Examples of species of bacteria used as compositions for administration to humans include Bifidobacterium bifidum; Escherichia coli, Lactobacillus acidophilus, Lactobacillus rhamnosus, Lactobacillus casei, and Lactobacillus johnsonii. Other species include Lactobacillus bulgaricus, Streptococcus thermophilus, Bacillus coagulans and Lactobacillus bifidus. Examples of strains of bacteria used as compositions for administration to humans include: B. infantis 35624, (Align); Lactobacillus plantarum 299V; Bifidobacterium animalis DN-173 010; Bifidobacterium animalis DN 173 010 (Activia Danone); Bifidobacterium animalis subsp. lactis BB-12 (Chr. Hansen); Bifidobacterium breve Yakult Bifiene Yakult; Bifidobacterium infantis 35624 Bifidobacterium lactis HN019 (DR10) Howaru™ Bifido Danisco; Bifidobacterium longum BB536; Escherichia coli Nissle 1917; Lactobacillus acidophilus LA-5 Chr. Hansen; Lactobacillus acidophilus NCFM Rhodia Inc.; Lactobacillus casei DN114-001; Lactobacillus casei CRL431 Chr. Hansen; Lactobacillus casei F19 Cultura Arla Foods; Lactobacillus casei Shirota Yakult Yakult; Lactobacillus casei immunitass Actimel Danone; Lactobacillus johnsonnii La1 (=Lactobacillus LC1) Nestlé; Lactobacillus plantarum 299V ProViva Probi IBS; Lactobacillus reuteri ATTC 55730 BioGaia Biologics; Lactobacillus reuteri SD2112; Lactobacillus rhamnosus ATCC 53013 Vifit and others Valio; Lactobacillus rhamnosus LB21 Verum Norrmejerier; Lactobacillus salivarius UCC118; Lactococcus lactis L1A Verum Norrmejerier; Saccharomyces cerevisiae (boulardii) lyo; Streptococcus salivarius ssp thermophilus; Lactobacillus rhamnosus GR-1; Lactobacillus reuteri RC-14; Lactobacillus acidophilus CUL60; Bifidobacterium bifidum CUL 20; Lactobacillus helveticus R0052; and Lactobacillus rhamnosus R0011.

The following U.S. patents, which are each incorporated herein by reference, disclose non-pathogenic bacteria which can be administered to individuals. U.S. Pat. No. 6,200,609; U.S. Pat. No. 6,524,574, U.S. Pat. No. 6,841,149, U.S. Pat. No. 6,878,373, U.S. Pat. No. 7,018,629, U.S. Pat. No. 7,101,565, U.S. Pat. No. 7,122,370, U.S. Pat. No. 7,172,777, U.S. Pat. No. 7,186,545, U.S. Pat. No. 7,192,581, U.S. Pat. No. 7,195,906, U.S. Pat. No. 7,229,818, and U.S. Pat. No. 7,244,424.

Accordingly the aspects of the invention, bacteria would first be provided with genetic material encoding a GCC agonist in a form that would permit expression le of the agonist peptide within the bacteria, either constitutively or upon induction by the presence of an inducer that would turn on an inducible promoter.

Some embodiments comprise inducible regulatory elements such as inducible promoters. Typically, an inducible promoter is one in which an agent, when present, interacts with the promoter such that expression of the coding sequence operably linked to the promoter proceeds. Alternatively, an inducible promoter can include a repressor which is an agent that interacts with the promoter and prevent expression of the coding sequence operably linked to the promoter. Removal of the repressor results in expression of the coding sequence operably linked to the promoter.

The agents that induce an inducible promoter are preferably not naturally present in the organism where expression of the transgene is sought. Accordingly, the transgene is only expressed when the organism is affirmatively exposed to the inducing agent. Thus, in a bacterium that includes a transgene operably linked to an inducible promoter, when the bacterium is living within the gut of an individual, the promoter may be turned on and the transgene expressed when the individual ingests the inducing agent.

The agents that induce an inducible promoter are preferably not toxic. Thus, in a bacterium that includes a transgene operably linked to an inducible promoter, the inducing agent is preferably not toxic to the individual in whose gut the bacterium is living such that when the individual ingests the inducing agent to turn on expression of the transgene the inducing agent dose not have any severe toxic side effects on the individual.

The agents that induce an inducible promoter preferably affect only the expression of the gene of interest. Thus, in a bacterium that includes a transgene operably linked to an inducible promoter, the inducing agent does not have any significant affect on the expression of any other genes in the individual.

The agents that induce an inducible promoter preferably are easy to apply or removal. Thus, in a bacterium that includes a transgene operably linked to an inducible promoter that is living in the gut of an individual, the inducing agent is preferably an agent that can be easily delivered to the gut and that can be removed, either by affirmative neutralization for example or by metabolism/passing such that gene expression can be controlled

The agents that induce an inducible promoter preferably induce a clearly detectable expression pattern of either high or very low gene expression.

In some preferred embodiments, the chemically-regulated promoters are derived from organisms distant in evolution to the organisms where its action is required. Examples of inducible or chemically-regulated promoters include tetracycline-regulated promoters. Tetracycline-responsive promoter systems can function either to activate or repress gene expression system in the presence of tetracycline. Some of the elements of the systems include a tetracycline repressor protein (TetR), a tetracycline operator sequence (tetO) and a tetracycline transactivator fusion protein (tTA), which is the fusion of TetR and a herpes simplex virus protein 16 (VP16) activation sequence. The Tetracycline resistance operon is carried by the Escherichia coli transposon (Tn) 10. This operon has a negative mode of operation. The interaction between a repressor protein encoded by the operon, TetR, and a DNA sequence to which it binds, the tet operator (tetO), represses the activity of a promoter placed near the operator. In the absence of an inducer, TetR binds to tetO and prevents transcription. Transcription can be turned on when an inducer, such as tetracycline, binds to TetR and causes a conformation change that prevents TetR from remaining bound to the operator. When the operator site is not bound, the activity of the promoter is restored. Tetracycline, the antibiotic, has been used to create two beneficial enhancements to inducible promoters. One enhancement is an inducible on or off promoter. The investigators can choose to have the promoter always activated until Tet is added or always inactivated until Tet is added. This is the Tet on/off promoter. The second enhancement is the ability to regulate the strength of the promoter. The more Tet added, the stronger the effect so now you can turn up or down an expression vector the way you turn up or down the volume on a radio.

Examples of inducible or chemically-regulated promoters include Steroid-regulated promoters. Steroid-responsive promoters are provided for the modulation of gene expression include promoters based on the rat glucocorticoid receptor (GR); human estrogen receptor (ER); ecdysone receptors derived from different moth species; and promoters from the steroid/retinoid/thyroid receptor superfamily. The hormone binding domain (HBD) of GR and other steroid receptors can also be used to regulate heterologous proteins in cis, that is, operatively linked to protein-encoding sequences upon which it acts. Thus, the HBD of GR, estrogen receptor (ER) and an insect ecdysone receptor have shown relatively tight control and high inducibility

Examples of inducible or chemically-regulated promoters include metal-regulated promoters. Promoters derived from metallothionein (proteins that bind and sequester metal ions) genes from yeast, mouse and human are examples of promoters in which the presence of metals induces gene expression.

IPTG is a classic example of a compound added to cells to activate a promoter. IPTG can be added to the cells to activate the downstream gene or removed to inactivate the gene.

U.S. Pat. No. 6,180,391, which is incorporated herein by reference, refers to the a copper-inducible promoter.

U.S. Pat. No. 6,943,028, which is incorporated herein by reference, refers to highly efficient controlled expression of exogenous genes in E. coli.

U.S. Pat. No. 6,180,367, which is incorporated herein by reference, refers to a process for bacterial production of polypeptides.

Other examples of inducible promoters suitable for use with bacterial hosts include the beta.-lactamase and lactose promoter systems (Chang et al., Nature, 275: 615 (1978, which is incorporated herein by reference); Goeddel et al., Nature, 281: 544 (1979), which is incorporated herein by reference), the arabinose promoter system, including the araBAD promoter (Guzman et al., J. Bacteriol., 174: 7716-7728 (1992), which is incorporated herein by reference; Guzman et al., J. Bacteriol., 177: 4121-4130 (1995), which is incorporated herein by reference; Siegele and Hu, Proc. Natl. Acad. Sci. USA, 94: 8168-8172 (1997), which is incorporated herein by reference), the rhamnose promoter (Haldimann et al., J. Bacteriol., 180: 1277-1286 (1998), which is incorporated herein by reference), the alkaline phosphatase promoter, a tryptophan (trp) promoter system (Goeddel, Nucleic Acids Res., 8: 4057 (1980), which is incorporated herein by reference), the P.sub.LtetO-1 and P.sub.lac/are-1 promoters (Lutz and Bujard, Nucleic Acids Res., 25: 1203-1210 (1997), which is incorporated herein by reference), and hybrid promoters such as the tac promoter. deBoer et al., Proc. Natl. Acad. Sci. USA, 80: 21-25 (1983), which is incorporated herein by reference. However, other known bacterial inducible promoters and low-basal-expression promoters are suitable.

U.S. Pat. No. 6,083,715, which is incorporated herein by reference, refers to methods for producing heterologous disulfide bond-containing polypeptides in bacterial cells.

U.S. Pat. No. 5,830,720, which is incorporated herein by reference, refers to recombinant DNA and expression vector for the repressible and inducible expression of foreign genes.

U.S. Pat. No. 5,789,199, which is incorporated herein by reference, refers to a process for bacterial production of polypeptides.

U.S. Pat. No. 5,085,588, which is incorporated herein by reference, refers to bacterial promoters inducible by plant extracts.

U.S. Pat. No. 6,242,194, which is incorporated herein by reference, refers to probiotic bacteria host cells that contain a DNA of interest operably associated with a promoter of the invention can be orally administered to a subject . . . .

U.S. Pat. No. 5,364,780, which is incorporated herein by reference, refers to external regulation of gene expression by inducible promoters.

U.S. Pat. No. 5,639,635, which is incorporated herein by reference, refers to a process for bacterial production of polypeptides.

U.S. Pat. No. 5,789,199, which is incorporated herein by reference, refers to a process for bacterial production of polypeptides.

U.S. Pat. No. 5,689,044, which is incorporated herein by reference, refers to chemically inducible promoter of a plant PR-1 gene.

U.S. Pat. No. 5,063,154, which is incorporated herein by reference, refers to a pheromone-inducible yeast promoter.

U.S. Pat. No. 5,658,565, which is incorporated herein by reference, refers to an inducible nitric oxide synthase gene.

U.S. Pat. Nos. 5,589,392, 6,002,069, 5,693,531, 5,480,794, 6,171,816 6,541,224, 6,495,318, 5,498,538, 5,747,281, 6,635,482 and 5,364,780, which are each incorporated herein by reference, each refer to an IPTG-inducible promoters.

U.S. Pat. Nos. 6,420,170, 5,654,168, 5,912,411, 5,891,718, 6,133,027, 5,739,018, 6,136,954, 6,258,595, 6,002,069 and 6,025,543, which are each incorporated herein by reference, each refer to an tetracycline-inducible promoters.

### Patient Populations

The compositions comprising appetite suppressing amounts of guanylyl cyclase C agonists may be used to suppress appetite in an individual who has been identified as desiring to suppress appetite. Such individuals include overweight individuals, individuals suffering from eating disorders such as compulsive or nervous eating, individuals wishing to conserve food, individuals unable to eat, and individuals wishing to reduce body fat and/or body weight. The compositions comprising appetite suppressing amounts of guanylyl cyclase C agonists may be used to suppress appetite in an individual who is not overweight in order to prevent weight gain. Appetite suppression with guanylyl cyclase C agonists may be used as adjunctive therapy in combination with psychological/psychiatric counseling and therapy, or in combination with other weight reduction or appetite suppressive compositions such as sibutramine, orlastat, Hoodia gordonii and extracts and components thereof.

A correlation between levels of proguanylin and/or prouroguanylin in the blood and levels of guanylin and/or uroguanylin in the intestine may be used to allow for the determination of proguanylin and/or prouroguanylin levels by a simple blood test which informs with respect to guanylin and/or uroguanylin levels in the intestine. In some embodiments, levels of proguanylin and/or prouroguanylin, the guanylin and uroguanylin precursors, respectively, that circulate in the blood can be determined and compared to the normal range of levels of proguanylin and/or prouroguanylin, i.e. the range of the amount of levels of proguanylin and/or prouroguanylin which is typically found in healthy, non-obese populations. If the level of levels of proguanylin and/or prouroguanylin is determined to be below either the median or lower limit of the range, an individual can be administered guanylin and/or uroguanylin or proguanylin and/or prouroguanylin. In some embodiments, levels of proguanylin and/or prouroguanylin in blood samples may be determined using antibody assays such as ELISA assays adapted to provided quatitative results. In some embodiments, levels of proguanylin and/or prouroguanylin in blood samples may be determined using blood samples obtained from an individual 5 minutes to 6 hours following ingestion of fat. In some embodiments, levels of proguanylin and/or prouroguanylin in blood samples may be determined using blood samples obtained from an individual 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, minutes, 170 minutes, 175 minutes, 180 minutes, 185 minutes, 190 minutes, 195 minutes, 200 minutes, 205 minutes, 210 minutes, 215 minutes, 220 minutes, 225 minutes, 230 minutes, 235 minutes, 240 minutes, 245 minutes, 250 minutes, 255 minutes, 260 minutes, 265 minutes, 270 minutes, 275 minutes, 280 minutes, 285 minutes, 290 minutes, 295 minutes, 300 minutes, 305 minutes, 310 minutes, 315 minutes, 320 minutes, 325 minutes, 330 minutes, 335 minutes, 340 minutes, 345 minutes, 350 minutes, 355 minutes, or 360 minutes following ingestion of fat. In some embodiments, levels of proguanylin and/or prouroguanylin in blood samples may be determined using blood samples obtained a period of time within a range selected from the group of every range that can be contain any two of the above listed 5 minute intervals, i.e. 5-10 minutes, 5-15 minutes, etc. In some embodiments, the blood test comprises ingesting a specific food prior to sample collection.

### Example

Guanylyl cyclase C (GCC) is an intestine-specific transmembrane receptor that synthesizes cGMP as its proximal effector. GCC has emerged as an important intermediary in signaling programs controlling appetite and body weight. C57/BL6 mice in which GCC signaling was eliminated exhibited excess body weight which was associated with adipocyte hypertrophy, an increase in subcutaneous and visceral adipose mass and obesity-related co-morbidities including hepatic steatosis, hyperinsulinemia, and glucose intolerance. Furthermore, GCC-deficient mice exhibited hyperphagia, and their excess weight gain was eliminated by restricting food intake to levels consumed by wild-type mice. The observed hyperphagia correlates with a defect in gut pathways regulating food intake, as GCC-deficient mice exhibit ˜50% reduction in the enteroendocrine satiety hormones cholecystokinin and glucagon-like peptide within the jejunum and ileum, respectively. These observations demonstrate a previously unrecognized role for GCC signaling in regulating appetite and body weight by modulating the function of enteroendocrine cells. This highlights a novel therapeutic paradigm in which oral hormone supplementation with GCC ligands could enhance enteroendocrine hormone levels and amplify nutrient satiety responses, thereby restricting appetite and defending against obesity.

GCC regulates body mass by controlling food consumption, as GCC-deficient mice are hyperphagic and their accelerated growth can be eliminated by restricting their diet to wild-type levels. GCC regulates satiety responses to nutrients. GCC signaling may control satiety by regulating the differentiation and/or function of enteroendocrine cells along the crypt-villus axis. The loss of GCC signaling produces a deficiency of satiety hormone production, leading to inadequate nutrient-stimulated satiety responses, resulting in hyperphagia and obesity.

1. A composition comprising a guanylyl cyclase C agonist formulated for sustained specific release of an amount effective to suppress appetite when administered orally to an individual.

2. The composition of claim 1 further comprising an anti-diarrheal compound or composition.

3. A composition comprising a guanylyl cyclase C agonist formulated for intravenous delivery in an amount effective to suppress appetite when administered intravenously to an individual.

4. The composition of claim 3 wherein the guanylyl cyclase C agonist is proguanylin and/or prouroguanylin.

5. A method of suppressing appetite in an individual who has been identified as desiring appetite suppression comprising the step of: administering to said individual a composition comprising a guanylyl cyclase C agonist in an amount of effective to suppress the appetite of the individual.

6. The method of claim 5 wherein the composition comprises the guanylyl cyclase C agonist formulated for sustained specific release of an amount effective to suppress appetite when administered orally to an individual.

7. The method of claim 6 wherein an anti-diarrheal compound or composition is administered in combination with the guanylyl cyclase C agonist formulated for sustained specific release.

8. The method of claim 6 wherein the composition further comprises an anti-diarrheal compound or composition.

9. The method of claim 5 wherein the guanylyl cyclase C agonist administered intravenously.

10. The method of claim 5 wherein the guanylyl cyclase C agonist is proguanylin and/or prouroguanylin.

11. The method of claim 5 wherein the individual has been identified as having a deficiency of proguanylin and/or prouroguanylin levels.

12. The method of claim 5 wherein the individual has been identified as being overweight.

13. A method of suppressing appetite in an individual who has been identified as desiring appetite suppression comprising the step of: administering to said individual a population of bacteria comprising bacteria which comprise a nucleic acid molecule that encodes guanylyl cyclase C agonist operably linked to regulatory sequences operable in said bacteria, wherein said bacteria is of a species that can live in a human colon as part of a human's gut flora.

14. A method of claim 13 wherein said bacterium is a gram negative bacterium.

15. A method of claim 13 wherein said bacterium is auxotrophic in a human large intestine.

16. A method of claims 13 wherein said regulatory sequences comprise an inducible promoter and said method comprises the step of administering an composition that activates the inducible promoter.
